SEQUENCE LISTING (8 sequences)

SEQ ID NO: 1 - protein, 276 aa, Sarcocystis neurona
MTRAVLLTFLTLCSARVSLVRAGAPPQATCANGETTVTKLGSSGALRIHCPNNFRLAPRAGNDAGQMQVYATAVAENPVNIRDVLPGASYLSVQNVPTLTVPQLPAKATSVFFHCQQQPDNQCFIQVEVAPAPRLGPNTCAALQSTIAFEVQQANETAVFSCGEGLAVFPQGSKALDEACSKEQALPSGAALAPKDGGLHLGFPQLPQQAMKICYICTNGGVQAEAAQRCEVRISVAANPDSGVPGANGAASLGAAARSASALGLALVAGAFLHFC

SEQ ID NO: 2 - DNA, 1132 bp, Sarcocystis neurona
gggaggtaagtgttggcggtaatgctgcatcattagggtcagacacgctgtccatctgtcattctcgccagaatgacgagggcggtgctgctgacgtttctgacactctgctccgccagagtgtcccttgtgagggccggagcgccgcctcaagcacgtgcgccaatggcgaaacgactgttactaagctcggcagctctggcgcactacgaatccactgcccaaataattttcgactcgcgccccgggctgggaatgacgccggtcagatgcaggtctatgcactgcggttgctgagaatcctgtaaacatacgagacgtcctgcccggcgcatcttacctctctgtacagaacgtcccgagggtcaccgtcccgcaattgcccgccaaagctacgagcgtcttttttcactgccagcagcaacccgacaaccaatgcttcatccagtcagctgcggcgagggacttgctgtgttcccgcaaggtagcaaagcgttggatgaagcctgctccaaagagcaggccctacccagtggcgccgctttagctccaaaggatggtgggctccaccttggttttcctcagcttcctcagcaggctatgaagatttgctatatttgtacgaatggtggtgtgcaggcagaggcggcccaacggtgtgaggttcgcatctccgtcgcagcgaacccagacggaagcgttccaggggctaacggagccgcctctctaggagctgccgcacgcagcgcctctgcgttagggttggctctcgttgcaggcgctttcttgcacttttgctaatcctgccgtgtagcgtctctggtggcccgccccacagatcctggttattcccacagcgtgtagcgtctctggtggcccgccccacagatcctggttacgcgttcagtaacgtgcctactgttccaaaacgggaaaatccgaagatgcaaaattcatccggtgcagcgtcccatgtgttcagttacgactggacgagtgtagtgagatggttttacatccattcgcagtgcagaggcgtgggctcgcatattttttttttgtagtgtgccgttgtagatccagcaagttaaatatgttagtcattttgagcgcctgttccacgtaggcggct

SEQ ID NO: 3 - DNA, 729 bp, Sarcocystis neurona
atgacgagggcggtgctgctgacgtttctgacactctgctccgccagagtgtcccttgtgagggccggagcgccgcctcaagcacgtgcgccaatggcgaaacgactgttactaagctcggcagctctggcgcactacgaatccactgcccaaataattttcgactcgcgccccgggctgggaatgacgccggtcagatgcaggtctatgcactgcggttgctgagaatcctgtaaacatacgagacgtcctgcccggcgcatcttacctctctgtacagaacgtcccgagggtcaccgtcccgcaattgcccgccaaagctacgagcgtcttttttcactgccagcagcaacccgacaaccaatgcttcatccagtcagctgcggcgagggacttgctgtgttcccgcaaggtagcaaagcgttggatgaagcctgctccaaagagcaggccctacccagtggcgccgctttagctccaaaggatggtgggctccaccttggttttcctcagcttcctcagcaggctatgaagatttgctatatttgtacgaatggtggtgtgcaggcagaggcggcccaacggtgtgaggttcgcatctccgtcgcagcgaacccagacggaagcgttccaggggctaacggagccgcctctctaggagctgccgcacgcagcgcctctgcgttagggttggctctcgttgcaggcgctttcttgcacttttgctaa

SEQ ID NO: 4 - DNA, 25 bp, Artificial sequence
  universal primer
gccggagctctgcagaattcnnnnn

SEQ ID NO: 5 - DNA, 33 bp, Sarcocystis neurona
ggggggatccgatgacgagggcgggtgctgctg

SEQ ID NO: 6 - DNA, 31 bp, Sarcocystis neurona
gggggatccttagcaaaagtgcaagaaagcg

SEQ ID NO: 7 - DNA, 32 bp, Sarcocystis neurona
acgaggatccgatgacgagggcggtgctgctg

SEQ ID NO: 8 - DNA, 33 bp, Sarcocystis neurona
acgaggatcccacggcaggattagcaaaagtgc

(19) United States
(12) Patent Application Publication
(10) Pub. No.: US 2006/0073524 A1
(43) Pub. Date: Apr. 6, 2006

(54) Definitive diagnosis of EPM in the horse

(76) Inventor: Siobhan Patricia Ellison, Fairfield, FL (US)

Correspondence Address: SIOBHAN PELLISON DVM PHD, PO Box 970, Fairfield, FL, 32634, US

(21) Appl. No.: 10/251,567

(22) Filed: Sep. 20, 2002

Related U.S. Application Data
(60) Provisional application No. 60/323,893, filed on Sep. 21, 2001.

(51) Int. Cl. G01N 33/569; G01N 33/53

(52) U.S. Cl. 435/7.22

(57) ABSTRACT

The present invention concerns detection of acute, chronic, or sub-acute equine protozoal myeloencephalitis by detecting antibodies in serum or CSF or the lymphoblastogenesis response to a recombinant protein that is folded in a conformationally correct state and is the immunodominant surface antigen of Sarcocystis neurona.

### FIELD OF THE INVENTION

The present invention concerns detection of human and non-human diseases caused by Apicomplexan parasites including but not limited to Sarcocystis neurona, Sarcocystis dasypus (syn S. neurona), Sarcocystis cruzi, Sarcocystis falcatula, Sarcocystis sp., Toxoplasma gondii, Neospora caninum, N. hughesi, Eimeria and Plasmodium.

### PRIOR ART

The following is a list of prior art references considered to be pertinent for the subsequent description:
1. Villegas E N, Lieberman L A, Mason N, Blass S L, Zediak V P, Peach R, Horan T, Yoshinaga S, Hunter C A. 2002. A role for inducible costimulator protein in the CD28-independent mechanism of resistance to Toxoplasma gondii. J Immunol July 15;169(2):937-43
2. Marsh A E, Barr B C, Lakritz J, Nordhausen R, Madigan J E, Conrad P A. Parasitol Res 1997. Experimental infection of nude mice as a model for Sarcocystis neurona-associated encephalitis. 83(7):706-11
3. Dame, J. B., MacKay, R. J., Yowell, C. A., Cutler, T. J., Marsh, A., and Greiner, E. C. 1995. Sarcocystis falcatula from passerine and psittacine birds: synonymy with Sarcocystis neurona, agent of Equine Protozoal Myeloencephalitis. J. Parasitol., 81(6):930-935.
4. Long M T, Mines M T, Knowles D P, Tanhauser S M, Dame J B, Cutler T J, MacKay R J, Sellon D C. 2002 Sarcocystis neurona: parasitemia in a severe combined immunodeficient (SCID) horse fed sporocysts. Exp Parasitol March ;100(3): 150-4
5. Dubey J. P., Davis S. W., Speer C. A., Bowman D. D., de Lahunta A., Granstrom D. E.,Topper M. J., Hamir A. N., Cummings J. F., and Suter M. M. 1991. Sarcocystis Neurona N. SP. (Protozoa: Apicomplexa), The etiologic Agent of Equine Protozoal myeloencephalitis. J. Parasitol., 77(2): 212-218.
6. Blythe, L. L., Granstrom, D. E., Hansen, D. E., Walker, L. L., Bartlett, J., and Stamper, S. 1997. Seroprevalence of antibodies to Sarcocystis neurona in horses residing in Oregon. JAVMA, 210(4): 525-528.
7. Bentz, B. G., Granstrom, D. E., Stamper, S. 1997. Seroprevalence of antibodies to Sarcocystis neurona in horses residing in a county of southeastern Pennsylvania. JAVMA, 210(4): 517-518.
8. Saville, W. J., Reed, S. M., Granstrom, D. E., Hincheliff, K. W., Kohn, C. W., Wittum, T. E., and Stamper, S. 1997. Seroprevalence of antibodies to Sarcocystis neurona in horses residing in Ohio. JAVMA, 210(4): 519-523.
9. Gray L. C., Magdesian, K. G., Sturges, B. K., Madigan, J. E. 2001. Suspected protozoal myeloencephalitis in a two-month-old colt. Vet Rec. Sep. 1, 2001;149(9):269-73.
10. MacKay, R. J. 1997. Serum antibodies to Sarcocystis neurona-half the horses in the United States have them! JAVMA, 210(4): 482-483.
11. Dubey, A. P. 1976. A review of Sarcocystis of Domestic Animals. JAVMA, 169 (10):1061-1078.
12. Dubey, J. P., 1986. Equine protozoal myeloencephalitis in a pony. JAVMA, 188:1311-1312.
13. Granstrom, D. E, Saville, W. J. 1998. Equine Protozoal Myeloencephalitis In: S. M. Reed and W. M. Bailey, eds. Equine Internal Medicine. Philadelphia, Pa.: W B Saunders Company, 486-491.
14. Fenger, C. K., Granstrom, D. E., Gajadhar, A. A., Williams, N. M., McCrillis, S. A., Stamper, S., Langemeier, J. L., Dubey, J. P. 1997. Experimental induction of equine protozoal myeloencephalitis in horses using Sarcocystis sp. sporocysts from the opossum (Didelphis virginiana). Vet Parasitol, 68:199-213.
15. O'Donoghue, P., Lumb, R., Smith, P., Brooker, J., Mencke, N. 1990. Characterization of monoclonal antibodies against ovine Sarcocystis spp. antigens by immunoblotting and immuno-electron microscopy. Vet. Immunol. Immunopathol., 24(1):11-25.
16. Marsh, A. E., Barr, B. C., Tell, L., Koski, M., Greiner, E., Dame, J.. and Conrad, P. A. 1997. In vitro cultivation and experimental inoculation of Sarcocystis falcatula and Sarcocystis neurona merozoites into budgerigars (Melopsittacus undulatus). J. Parasitol., 83(6): 1189-1192.
13. Dubey J R, Rosypal A C, Rosenthal B M, Thomas N J, Lindsay D S, Stanek J F, Reed S M Saville W J. 2001. Sarcocystis neurona infections in sea otter (Enhydra lutris): evidence for natural infections with sarcocysts and transmission of infection to opossums (Didelphis virginiana). J Parasitol December;87(6):1387-93.
14. Rosypal A C, Lindsay D S, Duncan R, Ansar Ahmed S, Zajac A M, Dubey J P. 2002 Mice lacking the gene for inducible or endothelial nitric oxide are resistant to sporocyst induced Sarcocystis neurona infections. Vet Parasitol February 4;103(4):315-21.
15. Dubey J R, Rosypal A C, Rosenthal B M, Thomas N J, Lindsay D S, Stanek J F, Reed S M, Saville W J. 2001. Sarcocystis neurona infections in sea otter (Enhydra lutris): evidence for natural infections with sarcocysts and transmission of infection to opossums (Didelphis virginiana). J Parasitol December;87(6):1387-93
16. Rosypal A C, Lindsay D S, Duncan R, Ansar Ahmed S, Zajac A M, Dubey J P. 2002 Mice lacking the gene for inducible or endothelial nitric oxide are resistant to sporocyst induced Sarcocystis neurona infections. Vet Parasitol February 4;103(4):315-21
17. Cheadle M A, Ginn P E, Lindsay D S, Greiner E C. 2002. Neurologic disease in gamma-interferon gene knockout mice caused by Sarcocystis neurona sporocysts collected from opossums fed armadillo muscle. Vet Parasitol January 3;103(1-2):65-9
18. Dubey J P, Lindsay D S, Kwok O C, Shen S K. 2001. The gamma interferon knockout mouse model for sarcocystis neurona: comparison of infectivity of sporocysts and merozoites and routes of inoculation. J Parasitol October;87(5):1171-3
19. Lindsay D S, Dubey J P. 2001. Determination of the activity of pyrantel tartrate against Sarcocystis neurona in gamma-interferon gene knockout mice. Vet Parasitol May 22;97(2):141-4
20. Dubey J P. 2001. Migration and development of Sarcocystis neurona in tissues of interferon gamma knockout mice fed sporocysts from a naturally infected opossum. Vet Parasitol February 26;95(2-4):341-51
21. Speer C A, Dubey J P. 2001. Ultrastructure of schizonts and merozoites of Sarcocystis neurona. Vet Parasitol February 26;95(2-4):263-71
22. Cheadle M A, Tanhauser S M, Scase T J, Dame J B, Mackay R J, Ginn P E, Greiner E C. 2001. Viability of Sarcocystis neurona sporocysts and dose titration in gamma-interferon knockout mice. Vet Parasitol February 26;95(2-4):223-31
23. Dubey J P, Mattson D E, Speer C A, Hamir A N, Lindsay D S, Rosenthal B M, Kwok O C, Baker R J, Mulrooney D M, Tornquist S J, Gerros T C. 2001. Characteristics of a recent isolate of Sarcocystis neurona (SN7) from a horse and loss of pathogenicity of isolates SN6 and SN7 by passages in cell culture. Vet Parasitol February 26;95(2-4): 155-66
24. Tenter, A. M., Johnson, M. R., Zimmerman, G. L. 1989. Differentiation of Sarcocyst species in European sheep by isoelectric focusing. Parasitol. Res. 76(2):107-114.
25. Sommer, I., Horn, K., Heydorn, A. O., Mehlhorn, H., Ruger, W. 1992. Acomparison of sporozoite and cyst merozoite surface proteins of Sarcocystis. Parasitol Res., 78:398-403.
26. Dubey, J. P., Kistner, T. P., and Callis, G. 1983. Development of Sarcocystis in mule deer transmitted through dogs and coyotes. Can. J Zool., 61:2904-2912.
27. O'Donoghue, P. J. and Ford, G. E. 1984. The asexual pre-cyst development of Sarcocystis tenella in experimentally infected specific-pathogen-free lambs. Int. J. Parasitol., 14(4):345-355.
28. Speer, C. A. and Dubey, J. P. 1981. An ultrastructural study of first and second generation merogony in the coccidian Sarcocystis tenella. J. Protozool., 28(4):424-431.
29. Johnson, A. J., Hildebrandt, P. K., and Fayer, R. 1975. Experimentally induced Sarcocystis infection in calves. Pathology Am. J. Vet. Res., 36(7):995-999.
30. Fayer, R. and Leek, R. G. 1979. Sarcocystis transmitted by blood transfusion. J Parasitol., 65(6):890-893.
31. Ellison, S. P., Omara-Opyeme, A. L., Yowell, Yowell, C. A., Marsh, A. E., Dame, J. B. 2002. Molecular characterization of a major 29 kDa surface antigen of Sarcocystis neurona. Int. J. Parasit. 32: 217-225.
32. Dubey, J. P., and Lindsay D. S. 1998. Isolation in immunodeficient mice of Sarcocystis neurona from opossum (Didelphis viriniana) feces and its differentiation from Sarcocystis falcatula. International Journal for Parasitology. 28:1823-8.
33. NAHMS. 2001. Equine Protozoal Myeloencephalitis (EPM) in the U.S. USDA:APHIS:VS, CEAH, National Animal Health Monitoring System. Fort Collins, Colo. #N312.0501.
34. Lindsay D S, Dykstra C C, Williams A, Spencer J A, Lenz S D, Palma K, Dubey J P, Blagburn B L. 2000 Inoculation of Sarcocystis neurona merozoites into the central nervous system of horses. Vet Parasitol. September 20;92(2):157-63.
35. Ellison, S. P., Greiner, E., Dame, J. B. 2000. In vitro culture and synchronous release of Sarcocystis neurona merozoites from host cells. Vet. Parasitol. 1982: 1-11.
36. Speer, C. A., Dubey, J. P. 2001. Ultrastructure of schizonts and merozoites of Sarcocystis neurona. Vet Parasitol. 95: 263-271.
37. Dubey, J. P., Mattson, D. E., Speer, C. A., Hamir, A. N., Lindsay, D. S., Rosenthal, B. M., Kwok, O. C., Baker, R. J., Mulrooney, D. M., Tornquist, S. J., Gerros, T.C. 2001. Characteristics of a recent isolate of Sarcocystis neurona (SN7) from a horse and loss of pathogenicity of isolates SN6 and SN7 by passages in cell culture. Vet Parasitol. February 26;95(2-4): 155-66
   The acknowledgement herein of any of the above references is to allow the reader to gain appreciation of the prior art. The acknowledgement should, however, not be construed as an indication that these references are in any way relevant to the issue of patentability of the invention as defmed in the appended claims.

Acknowledgement of the above references will be made by indicating the number from the above list.

### BACKGROUND OF THE INVENTION

Apicomplexan parasites are highly successful partially due their ability to manipulate the host's immune system in favor of the parasite. In addition to antibody response, long-term resistance to these infections is possibly dependent on the development of parasite-specific T cells that produce IFN-ganuma. Several molecules, such as CD28, are costimulatory and result in activation of T cells, although CD28-independent mechanisms also regulate T cell responses during infection. The identification of other inducible costimulator proteins (ICOS) and the ability to regulate the production of IFN-gamma suggested that this mechanism may be involved in the CD28-independent activation of T cells required for resistance to T. gondii. [1] Villegas and co-workers found when both costimulatory pathways were blocked in T. gondii infections, there was an additive effect that resulted in almost complete inhibition of IFN-gamma production. Further, blocking both pathways led to increased susceptibility of CD28(−/−) mice to T. gondii associated with reduced serum levels of IFN-gamma, increased parasite burden, and increased mortality compared with the control group.

Apicomplexan parasites such as Toxoplasma, Neospora, and Sarcocystis cause diseases in immunologically privileged compartments such as the brain, spinal cord or fetal tissues. Mammals cannot easily stimulate an immune response to control parasite invasion of these compartments. Equine Protozoal Myeloencephalitis (EPM) is the leading infectious neurologic equine disease in the Western Hemisphere. While the symptoms and effects of EPM have been recognized since the 1970's, it was not until 1991 that the protozoan parasite that causes EPM was isolated and cultured from a horse and given the name Sarcocystis neurona [2].

Although Dame and co-workers implicated S. falcatula as the etiologic agent of EPM based on molecular data, Sarcocystis neurona (S. neurona), recently recognized as S. dasypus (syn. S. neurona), is the agent that has been isolated from the CSF of diseased horses.[3] Sarcocystis neurona cycles naturally between opossums and armadillos/raccoons while S. falcatula cycles between opossums and cowbirds or grackles. Recent investigations indicate that the feces of the opossum (the definitive host) may be the source of the infection for horses. Thus, the horse is an aberrant host, becoming exposed when it consumes infectious material from opossum feces. An aberrant host is a dead-end host, as infectious forms of the parasite are not passed from horse to horse or from infected horse to a definitive or true intermediate host. Incidence of EPM is likely greatest in areas with high opossum populations. EPM appears to have a sporadic distribution, although outbreaks have been reported on farms in Kentucky, Ohio, Indiana, Michigan and Florida [4-6].

In the horse, S. neurona produces clinical signs of disease as a result of merozoites that make their way to the brain and spinal cord, where they proliferate and cause clinical disease. Clinical signs of EPM do not develop until the organism has crossed the blood brain barrier and is within the central nervous system. These signs include weakness, muscle atrophy, spinal ataxia, or “wobbling” and/or head tilt with asymmetry of the face (e.g., eyelid, ear, or lip). A severely EPM-affected horse may go down and be unable to rise. Lameness not traceable to orthopedic disease or any combination of the above signs may occur in early or less severe infections. In most cases, an affected horse is bright and alert with a normal appetite, hematological and biochemical blood values are usually in the normal range.

Originally, the diagnosis of EPM was based on the presence of antibodies to S. neurona in serum detected by immunoblot. The immunoblot used to detect serum antibodies uses antigen that is first separated by size, though it is now known that a positive serum test using this complex antigen preparation cannot be used to make a diagnosis. There are several reasons why reactive antibodies to these antigens are not diagnostic. First, the antigens at key molecular weights represent several proteins, some of which are cross reactive with other species of sarcocystis. Also, the immunodominant antigen of S. neurona reacts with serum and CSF antibodies derived from horses when the antigen is folded in the proper conformation. For the protein to be folded correctly the antigen must be in a non-reduced state. Therefore, an immunoblot test with a positive serum test simply indicates exposure to the parasite, not necessarily presence of the disease. Cerebral spinal fluid (CSF) testing by immunoblot is now believed to be the most useful test to assist in the diagnosis of this disease in a live horse [10]. The most promising test is a single, specific immunodominant protein used as antigen (rSnSAG1) in an enzyme linked immunosorbent assay (ELISA). This antigen is a recombinant protein made in E. coli that has the same specificity for equine antibodies against S. neurona as the native antigen. [31] Additionally, serial testing using the rSNSAG1 ELISA with serum or CSF from suspect clinical cases of EPM may predict acute disease or clinical outcome. Cases of acute EPM from experimentally induced infections (Ellison animal model of EPM in the horse 2002) indicate the serum titer before (day 0) and after challenge (day 32) was increased during infection and titer reduced after successful treatment (Table 1). In this study the challenged animals were treated prior to day 0 to prevent infection. All animals evidenced mild to moderate signs of EPM (Table 2) as evaluated by three evaluators blinded as to treatment. Additionally, all animals developed a CSF titer as shown in Table 3.

Surveys (using a positive serum test to immunoblotted S. neurona antigens to indicate exposure to the parasite) have revealed that approximately fifty percent (50%) of the horses in the surveyed areas have been exposed to S. neurona [3-5,7]. However, a positive test result on the immunoblot test does not necessarily indicate the presence of an active form of the disease. The incidence of the active disease appears to be much lower than the seroprevalence since less than 1% of seropositive horses are clinically affected [7]. Survey results with rSnSAG1 used as an antigen in ELISA testing have revealed that as few as 8% of animals have positive serum test results when serum was used from a small group of clinically normal horses in Florida (Table 1) when a titer of ≦50 was considered a negative test result.

It is unknown how many horses contract the disease and exhibit mild neurologic abnormality. The economic significance of mild neurologic impairment impacts the performance animal. Of animals recognized as clinically affected, 30-40% reportedly fails to respond to current therapy, and some of these animals die [7]. The current perception of treatment failure may be due to the chronic nature of the infection when it is recognized. It is probable that an aspect of the disease and its clinical signs rest with the inflammatory reaction to the proliferating merozoites. Success of treatment is enhanced it infection is found prior to CNS damage. Damage can occur as a direct result of merozoites entering and proliferating in the CNS. Additional damage can occur due to the inflammatory response to parasites in the CNS.

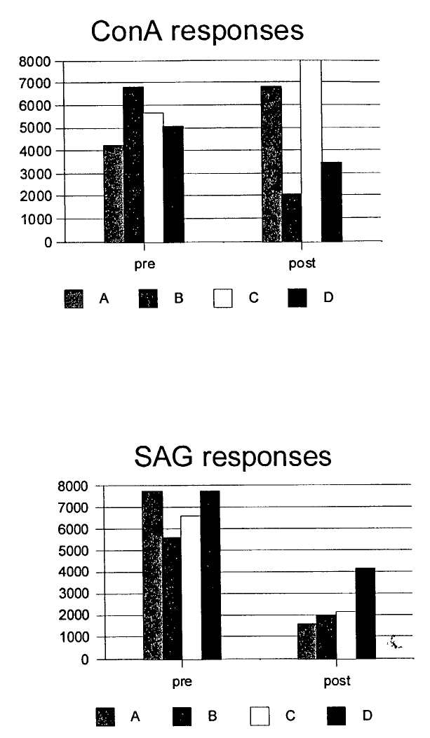

Initial inflammatory cytokines, prior to merozoites entering the CNS, are important in the outcome of disease. A test that measures the interferon gamma response to rSnSAG1 using RT-PCR indicates that interferon gamma is down regulated by the parasite in initial infection (FIG. 1). The rSnSAG1 interferon gamma test can detect depression of this inflammatory response at the time merozoites are transported in the peripheral circulation prior to entry into the CNS. The uses of these tests are important evaluators of clinical disease in the horse.

In summary, the recognition of infection of a host by an apicomplexan parasite prior to its entry into the CNS is critical to a positive treatment outcome. One of the necessary first steps for successful infection is the down regulation of host interferon gamma response followed by an antibody response; the detection of an antibody response and depression of the host's interferon gamma response can be achieved using recombinant protein technology in RT-PCR and ELISA testing.

### DETAILED DESCRIPTION OF THE INVENTION

As set forth above, the present invention is directed to a unique discovery that enables the identification of acute, chronic, or sub-clinical disease caused by central nervous system infection of a mammal by Sarcocystis neurona that is not possible with immunoblot. A significant finding unique to this invention is the use of a single antigen, the immunodominant surface antigen of S. neurona that has been prepared in a correctly folded state to allow conformational epitopes to form that are important for the binding of diagnostic antigens. A major advantage of the techniques of the present invention is that it can be used to identify Apicomplexan diseases in mammals that can either be an intermediate non-human mammalian host or a non-human aberrant intermediate host. Apicomplexan diseases for which the diagnostic tests are useful include but are not limited to EPM caused by S. neurona, S. dasypus, and S. falcatula. Mammals useful for this test include but are not limited to equines, cats, opossums, armadillos.

In accordance with the invention the diagnostic tests using recombinant protein as an immunodominant Sarcocystis antigen that is not shared by any other Apicomplexans including, but not limited to Toxoplasma and Neospora. The preferred said immunodominant Sarcocystis antigen for the present invention are, including but not limited to, recombinant surface protein antigens of S. neurona SAG 1 and SAG2 that elicit an immune response during infection with the native parasite. Additional recombinant proteins that can be used herein are those said immunodominant Sarcocystis antigens that correspond to surface antigens from the Apicomplexan parasite Sarcocystis neurona or Sarcocystis falcatula that stimulate an immune reaction at initial infection These recombinant proteins that represent the said immunodominant Sarcocystis antigen can be cultured in vitro by those familiar with the art.

The diagnostic tests of Apicomplexan disease described herein generally comprises, but is not limited to the steps of: 1) coating a microtiter plate with recombinant protein representing said immunodominant Sarcocystis antigen; 2) blocking the unbound sites on the plate; 3) incubating equine serum or CSF with the said immunodominant Sarcocystis antigen and 4) optionally, detecting the presence of antibodies bound to the said immunodominant Sarcocystis antigen that represents infection by the Apicomplexan from the infected host by reaction with a secondary, labeled, antibody. This detecting antibody can be, but not limited to alkaline phosphatase, horseradish peroxidase, or fluroescent label. A second method described herein generally comprises, but is not limited to the steps of: 1) incubating isolated peripheral equine lymphocytes with said immunodominant Sarcocystis antigen; 2) harvesting the mRNA from the stimulated lymphocytes; 3) using RT-PCR to detect the lymphoblastogenic response from the stimuated lymphocytes.

Critical to the present invention is the production of the said immunodominant Sarcocystis antigen in an active form. In accordance with the invention, the said immunodominant Sarcocystis antigen retains activity as long as the amino acid interactions, found in all Apicomplexan organisms surface antigens, is produced in the active form. Appropriate to the production of active antigen the antigen is first reduced, purified, and then refolded.

The described invention is useful to detect infections in experimental animals or natural infections. Rodents inoculated S. neurona are useful, in accordance with the invention, as a non-host animal model for vertical transmission to a fetus or developing embryo. Horses inoculated with S. neurona are useful, in accordance with the invention, as an animal model for EPM in the horse for testing drug and prophylactic therapies. Finally, felines inoculated with such S. neurona are useful in accordance with the invention as an animal model for EPM investigations. Such experimental investigations can be advantageously used, for example, for screening or determining efficacy of drugs for treatment or prophylaxis of diseases such as EPM. Finally, the animals inoculated with such parasite and infections detected with the said invention are useful to elucidate the pathophysiology of disease.

More specifically described is a method for the diagnosis of EPM in the horse, said method demonstrating serum and CSF antibody production against S. neurona and a method for detecting the manipulation of interferon gamma during acute infection by S. neurona. As part and parcel of the invention, there is presented herein the recognition of the mechanism of these tests as a problem transcended. The present invention describes methods to identify acute infection using specific said immunodominant Sarcocystis antigens.

Diagnostic products produced using the described tests include but are not limited to test systems that comprise one or more antibodies, one or more antigens, antibodies or homologus DNA/RNA material in the mammal. Test systems included but are not limited to ELISA, immunoblot, western blot, agglutination, hemagglutination, latex agglutination, PCR detection, etc.

The diagnostic test that determines whether the presence or absence of interferon gamma specific in the lymphoblastogenic response is stimulated in lymphocytes is described. Novel to this assay is the discovery that horses infected with S. neurona show a decrease in stimulation of lymphocytes in the lymphoblastogenic response during acute infection. Also novel is the discovery that the antibody response in acute infection can be used to predict outcome of treatment during the treatment period.

The invention will now be illustrated by some non-limiting, specific embodiments described in the following examples.

### EXAMPLE 1

### Using ELISA for the Detection of Antibodies in Response to S. neurona Infection in the Horse

The most commonly accepted methods of diagnosis of S. neurona infection are clinical signs in conjunction with antibodies in the serum and cerebrospinal fluid. Necropsy with subsequent-postmortem histologic examination of neurologic tissues or culture of the organism from the central nervous system of the horse are the most specific method of diagnosis of EPM, but used rarely. Serology and spinal fluid analysis demonstrating antibodies against S. neurona are used commonly to diagnosis infection but show that more than 50% of horses have antibodies when native antigens of S. neurona are used in the assay. These results, when taken together, are interpreted that serology can be used to document exposure to parasites thought to cause EPM, horses apparently can be exposed frequently without ever developing clinical disease.¹⁴ The most common clinical signs reportedly seen among horses with EPM are ataxia, limb weakness, and lameness. We experimentally induced EPM in 49 horses and measured the antibody responses. Samples from experimentally infected horses were used and the assays run as follows.

Serum antibodies were screened at a dilution of 1:50 at two fold dilutiions until an endpoint titer was determined and CSF was screened undiluted and at two fold dilutions until an endpoint was determined. Serum and CSF were diluted for titration using PBST containing 1% BSA.

### Production of Antigen

Recombinant protein was purified from an expression vector as previously described using an affinity column as per manufacturer recommendations (Novagen). The total protein was determined using BCA protein detection kit (Pierce). The protein was diluted to 10 micrograms/ml using carbonate buffer (Sigma). An aliquot of 50 microliters was added to each well of a 96 well plate and incubated overnight at 4 C.

### ELISA

Immunosorp Maxi plates were blocked using 100 microliters 1% BSA in PBST (PBS, 0.05 Tween 20) that was added to each well of a 96 well plate and incubated 30 min room temperature. The plate was washed using PBST and 50 micro liters rSAG1 (10 μg/ml) was added and incubated overnight at 4 C. The plates were washed 3 times in PBST and either serum or CSF added. The plates were incubated two hours at room temperature and then washed three times with PBST. Fifty microliters of secondary antibody, goat anti horse IgG-alkaline phosphatase (Sigma) diluted 1:3000 was added to each well. The plates were incubated 30 minutes to 1 hour and then washed 3 times with PBST. The substrate, para-nitrophenyl phosphate (Sigma) was prepared in carbonate buffer and incubated 30 or 60 minutes at room temperature. The plate was read at 405 nM on

We have demonstrated that horses with S. neurona in the CNS with dramatic changes in the CSF did not evidence lameness until seven days later. Using 49 experimental EPM infections in horses we have determined several trends. The use of an immunodominant Sarcocystis antigen in recombinant form as an antigen has revealed that less than 8% of normal horses tested have circulating antibodies and therefore exposure is much less than previously projected. Additionally, horses produce serum antibodies that relate to clinical signs and, amazingly, a drop in antibody titer parallels clinical improvement. The detection of antibodies in the CSF also occurs in conjunction with clinical signs but CSF antibody titer did not respond as quickly as serum antibodies. The determination that the CSF does contain antibodies to S. neurona is an important diagnostic parameter; however, the serial determination of serum antibodies is an important discovery. The detection of these subtle antibody trends is due to the specificity of the active recombinant protein to the immunodominant Sarcocystis antigen.

### EXAMPLE 2

### Development of a Diagnostic Test Using Experimentally Derived Cases of EPM

Using RT-PCR for the Detection of IFNγ Production in Response to rSnSAG1 Antigen

The most commonly accepted methods of diagnosis of S. neurona infection are clinical signs in conjunction with antibodies in the serum and cerebrospinal fluid. Necropsy with subsequent postmortem histologic examination of neurologic tissues or culture of the organism from the central nervous system of the horse are the most specific method of diagnosis of EPM, but used rarely. The most common clinical signs reportedly seen among horses with EPM are ataxia, limb weakness, and lameness. We have demonstrated that horses with S. neurona in the CNS did not evidence lameness until seven days later. In one study, the equine model of EPM disease described was used to determine whether the presence or absence of Interferon gamma (IFNγ) specific for SnSAG1 as assayed by RT PCR indicated EPM. The Pilot horse and four additional horses were tested prior to infection, after infection and after development of clinical signs of EPM using the IFNγ specific for rSnSAG1 (the immunodominant surface antigen of S. neurona) as assayed by RT PCR. White blood cells from each animal were tested once a week for IFNγ stimulation by rSnSAG1. Serum samples from each horse were also obtained each week and were evaluated for IgG to recombinant protein that represents the immunodominant surface antigen of S. neurona by direct ELISA. The procedure for the IFNγ assay was as follows. Recombinant protein (rSnSAG1) was prepared from E. coli or yeast by art known techniques, diluted, and added to each well of a 96 well plate to yield 2 μg recombinant protein per well. The plate was incubated overnight at 4° C. and then washed in sterile phosphate buffered saline (PBS). Fresh lymphocytes from each horse were collected as described previously. Lymphocytes were added to a 96 well culture plate that had been coated with 1 μg rSnSAG1 and incubated for 24, 48, 72, and 96 hours. At each time point the stimulated lymphocytes were subjected to a lymphoblastogenesis response as measured by uptake of a label detected as counts per minute. Additionally, at each time point the stimulated lymphocytes were removed to a microfuge tube containing 50 μl RNAlater (Ambion). RNA was isolated using 200 μl Trizol reagent and using a phenol-chloroform extraction procedure. Following a second extraction with phenol, chloroform, and isoamyl alcohol, the RNA was precipitated using 20 μl ammonium acetate (3M, pH 4.2) and 250 μl isopropyl alcohol and incubated at −20° C. for one hour or overnight. The RNA was pelleted by centrifugation for 30 min in a microfuge and the pellet washed in 95% EtOH. The pellet was resuspended in 50 μl nuclease free water. A reaction mixture of 5 μl RNA, 3 μl 12 mM dNTP's, 1 μl oligo dT primer, and 7 μl water was heated to 80° C. for three minutes and cooled on ice. Each tube received 4 μl 5× buffer, 1 μl reverse transcriptase, and 1 μl RNase inhibitor. The reaction was incubated at 42° C. for 1 hour. The cDNA was cooled on ice and 5 μl aliquot added to a PCR reaction containing 3 μl 12 mM dNTP's, 5 μl 10× buffer, and 1 μl each forward and reverse equine IFNγ primer (5′aacctgaggaagcggaagaggagt3′ forward, 3′ttggactccttcgccttctcct5′ reverse), 1 U TAQ polymerase, and 35 μl water. The amplification conditions were 94° C. for 4 min followed by 35 cycles: 94° C. 45 sec, 60° C. 45 sec, and 72° C. 2 min. The 487 bp amplico representing the equine IFNγ mRNA sequence was visualized by electrophoresis using 2% agarose. A sample was determined as positive if the 487 bp amplicon was present and a sample was determined negative if there was no amplification using the IFN primers. The results for horses with EPM have consistently been negative while horses that are not infected with S. neurona or have been successfully treated for S. neurona EPM are positive for the amplicon in this test. The results of testing the high dose horse, medium dose horse and low dose horse as well as the control horse made it clear that all horses were able to stimulate interferon γ production prior to being infected and all but the control horse lost the ability to stimulate interferon γ to rSnSAG1 after infection. It is also important to note that the loss of the interferon γ response to rSnSAG1 was dose related over time. The high and mid-dose horses lost the ability to respond sooner than the low-dose horse. An additional 24 horses were challenged with S. neurona and the lymphoblastogenesis measured to both Con A (control) and rSAG1 the immunodominant antigen of S. neurona. It was found that horses could respond to ConA before and after challenge but when lymphoblastogenic responses were measured to rSAG1, the mean counts per minute were depressed post challenge. The production of interferon gamma after infection was absent in horses that were experimentally challenged.

It will be appreciated that the instant specification and claims are set forth by way of illustration and not limitation, and that various modifications and changes may be made without departing from the spirit and scope of the present invention.

TABLE 1
ELISA titer Serum

| ID | Day 0 | Day 32 | Day 64 |
|---|---|---|---|
| 1 | <50 | 400 | 100 |
| 2 | <50 | 200 | <50 |
| 3 | 50 | <50 | <50 |
| 4 | 50 | 100 | <50 |
| 5 | 100 | 100 | 200 |
| 6 | 50 | 50 | 100 |
| 7 | 50 | 800 | 1600 |
| 8 | 50 | 100 | 200 |
| 9 | 50 | 100 | 400 |
| 10 | 50 | 50 | 200 |
| 11 | 50 | 50 | <50 |
| 12 | 50 | 50 | 200 |
| 13 | 50 | <50 | <50 |
| 14 | 50 | <50 | 400 |
| 15 | <50 | 200 | 50 |
| 16 | <50 | <50 | 100 |
| 17 | 200 | 400 | 400 |
| 18 | <50 | 400 | 400 |
| 19 | 50 | 50 | 100 |
| 21 | 50 | 400 | 200 |
| 24 | 50 | 400 | 200 |
| 25 | <50 | 400 | 100 |
| 27 | 50 | 200 | 50 |
| 28 | 50 | 50 | <50 |

TABLE 2
Ataxia score

| | | | Ataxia |
|---|---|---|---|
| ID | Day 0 | Day 32 | Day 64 |
| 1 | 0 | 2 | 0 |
| 2 | 0 | 1 | 2 |
| 3 | 0 | 0 | 0 |
| 4 | 0 | 0 | 0 |
| 5 | 0 | 0 | 0 |
| 6 | 0 | 2 | 0 |
| 7 | 0 | 0 | 0 |
| 8 | 0 | 2 | 0 |
| 9 | 0 | 1 | 2 |
| 10 | 0 | 0 | 0 |
| 11 | 0 | 0 | 0 |
| 12 | 0 | 0 | 1 |
| 13 | 0 | 2 | 1 |
| 14 | 0 | 0 | 0 |
| 15 | 0 | 0 | 2 |
| 16 | 0 | 0 | 1 |
| 17 | 0 | 2 | 1 |
| 18 | 0 | 3 | 2 |
| 19 | 0 | 0 | 1 |
| 21 | 0 | 0 | 0 |
| 24 | 0 | 2 | 2 |
| 25 | 0 | 0 | 0 |
| 27 | 0 | 0 | 1 |
| 28 | 0 | 1 | 2 |

TABLE 3
Elisa titer CSF

| ID | Day 0 | Day 32 | Day 64 |
|---|---|---|---|
| 1 | 0 | 2 | 2 |
| 2 | 0 | 2 | 1 |
| 3 | 0 | — | 3 |
| 4 | 0 | 2 | 6 |
| 5 | 0 | — | 2 |
| 6 | 0 | — | 6 |
| 7 | 0 | 2 | 3 |
| 8 | 0 | 1 | 2 |
| 9 | 0 | 2 | 2 |
| 10 | 0 | 2 | 2 |
| 11 | 0 | 2 | 2 |
| 12 | 0 | 2 | 6 |
| 13 | 0 | 2 | 2 |
| 14 | 0 | — | 2 |
| 15 | 0 | — | 3 |
| 16 | 0 | 2 | 2 |
| 17 | 0 | — | 12 |
| 18 | 0 | 2 | 6 |
| 19 | 0 | 1 | 2 |
| 21 | 0 | 1 | 2 |
| 24 | 0 | 1 | 2 |
| 25 | 0 | 2 | 2 |
| 27 | 0 | 2 | 3 |
| 28 | 0 | 2 | 2 |

What is claimed is:

1. A method of detecting the presence of antibodies specifically immunoreactive with an equine Sarcocystis antigen in a biological sample, the method comprising contacting the sample with an isolated immunodominant Sarcocystis antigen, thereby forming an antigen/antibody complex, and detecting the presence or absence of the said antigen/antibody complex, wherein the antigen has an amino acid sequence that is at least 90% identical to SEQ ID NO 10.

2. The method of claim 1, wherein the immunodominant Sarcocystis antigen has the amino acid sequence shown in SEQ ID NO: 10.

3. The method of claim 1, wherein the biological sample is equine serum.

4. The method of claim 1, wherin the biological sample is equine CSF.

5. The method of claim 1, wherein the immunodominant antigen is immobilized on a solid surface.

6. The method of claim 1, wherein the antigen/antibody complex is detected using a labeled anti-equine antibody.

7. The method of claim 6, wherein the labeled anti-equine antibody is selected from the group consisting of alkaline phosphatase, horse radish peroxidase, flurocene isothiocyonate, and luceriferase.

8. The method of claim 1, wherein which the equine Sarcocystis antigen is the immunodominant surface antigen of Sarcocystis selected from the group consisting of S. neurona and S. falcatula.

9. A method of detecting the presence of production of equine interferon gamma specifically stimulated with an equine Sarcocystis antigen in a biological sample, the method comprising
a. contacting the sample with an isolated immunodominant Sarcocystis antigen,
b. thereby eliciting a lymphoblastogenic response resulting in with production of interferon gamma, and
c. detecting the presence or absence of the said interferon gamma.

10. The method of claim 9, wherein the isolated immunodominant antigen has an amino acid sequence that is at least 90% identical to SEQ ID NO 10.

11. The method of claim 9, wherein in which the biological sample is equine lymphocytes.

12. The method of claim 9, wherein which the lymphoblastogenic response stimulated lymphocytes is assayed using an RT-PCR probe.

13. The method of claim 12, wherein the RT-PCR probe comprises a primer having a sequence that is at least 90% identical with SEQ ID NO 20.

14. The method of claim 9, wherein the lymphoblastogenic response is stimulated lymphocytes is assayed using RT-PCR and the amplicon is detected by electrophoresis.

15. The method of claim 9 wherein the lymphoblastogenic response is stimulated lymphocytes are assayed using RT-PCR using primers having a sequence that is at least 90% identical to SEQ ID NO 20 and that additionally having have a biotin label.

16. The method of claim 9, wherein the stimulated lymphocytes the lymphoblastogenic response is assayed using RT-PCR and the amplicon is detected by avadin/biotin complex.

17. The method of claim 9, wherein the stimulated lymphocytes are the lymphoblastogenic response is assayed using RT-PCR and the amplicon is detected by a flurocein label.

18. The method of claim 9, wherein which the lymphoblastogenic response is stimulated lymphocytes is assayed using RT-PCR and the amplicon is detected by luceriferase.

19. The composition of an equine interferon gamma primer comprising a sequence that is at least 90% identical to SEQ ID NO 20.

20. A composition comprising an immunodominant Sarcocystis antigen conformationally folded to effect binding of antibodies in serum and CSF of a mammal.
